(19) United States
(12) Patent Application Publication
(10) Pub. No.: US 2010/0196905 A1
(43) Pub. Date: Aug. 5, 2010

(54) Adult Stem Cell Assays

(75) Inventors: Amy K. McNulty, San Antonio, TX (US); Kathleen L. Derrick, San Antonio, TX (US); Diwi Allen, San Antonio, TX (US)

Correspondence Address: Finnegan/KCI, 901 New York Avenue, NW, Washington, DC, 20001, US

(73) Assignee: KCI Licensing, Inc.

(21) Appl. No.: 12/694,932

(22) Filed: Jan. 27, 2010

Related U.S. Application Data
(60) Provisional application No. 61/148,667, filed on Jan. 30, 2009.

(51) Int. Cl. C12Q 1/68; G01N 33/53

(52) U.S. Cl. 435/6; 435/7.21; 435/7.24

(57) ABSTRACT

Provided are methods for quantifying adult stem cells in a test sample, methods of quantifying adult stem cells in a patient, and methods for quantifying differentiated cells in a test sample. Also provided are kits for quantifying adult stem cells or differentiated cells in a sample.

This application claims priority to U.S. Provisional Patent Application No. 61/148,667, filed Jan. 30, 2009, which is incorporated herein by reference in its entirety for all purposes.

### FIELD

Methods of quantifying adult stem cells in a sample are provided. Methods of quantifying adult stem cells in a patient are also provided. Kits for quantifying adult stem cells in a sample are further provided.

### BACKGROUND

Stem cells are progenitor cells that have the capacity to self-renew and differentiate into various cell lineages. There are two broad classes of stem cells, embryonic stem cells and adult stem cells. Embryonic stem cells are found in embryonic tissue and have the capacity to differentiate into any mature cell type. Thompson et al., (1998) Science 282(5391):1145-1147. Adult stem cells are found in adult tissues and have the capacity to differentiate into cell types of the tissue from which they originated, and possibly cell types from other tissues. See, e.g., Filip et al., (2004) J. Cell. Mol. Med. 8(4):572-577.

The ability of stem cells to differentiate into multiple cell lineages highlights their potential for use in regenerative medicine (see, e.g., Bajada et al., J. Tissue Eng. Regen. Med. (2008) 2(4):169-183), tissue engineering (see e.g., Tae et al., Biomed. Mater. (2006) 1(2):63-71), and wound healing (see, e.g., Branski et al., Burns (2008)). Adult stem cells may have comparable therapeutic potential to embryonic stem cells without posing many of the ethical dilemmas of embryonic stem cells. See, e.g., Hombach-Klonisch et al., J. Mol. Med. (2008) 86(12):1301-1314, Mauron et al., Clin. Pharpmacol. Ther. (2007) 82(3):330-333.

Despite the promise of adult stem cells, they constitute a very small percentage of cells within adult tissue, thus making them difficult to identify and quantify in tissue samples. For example, mesenchymal stem cells make up only about 0.001% to 0.01% of total bone marrow cells. Pittenger et al., Science (1999) 284(5411):143-147. To date, there have been no specific and unique set of stem cell markers identified, making adult stem cells difficult to isolate. For example, although markers such as NANOG, SOX2, and POU5F1 indicate pluripotency, these markers are not specific to mesenchymal stem cells or hematopoietic stem cells as they are also expressed on embryonic stem cells. See, e.g., Adewumi et al., Nat. Biotechnol. (2007) 25(7):803-816, Yu et al., Science (2007) 318(5858):1917b-1920, Takahashi et al., Cell (2007) 131(5):861-872. Furthermore, methods of identifying stem cells often require obtaining cells from a tissue of interest, enriching and expanding the cells of interest in vitro, and detecting expression of cell surface antigens. However, the culture conditions may alter expression of cell surface antigens, decreasing the reliability and reproducibility of such identification methods. Dazzi et al., Blood Rev. (2005) 20(3):161-171. Thus, methods for identifying and quantifying adult stem cells, including hematopoietic stem cells and mesenchymal stem cells, are needed.

### SUMMARY

Provided herein are methods for quantifying adult stem cells in a test sample. Also provided are methods for quantifying adult stem cells in a patient. The methods include quantifying the expression of at least one adult stem cell marker in the test sample. The methods also include determining the amount of the at least one adult stem cell type in the test sample by comparing the expression of the at least one adult stem cell marker in the test sample to the expression of the at least one adult stem cell marker in a control sample containing a known amount of the at least one adult stem cell type.

In some embodiments, the at least one adult stem cell type is selected from mesenchymal stem cells and hematopoietic stem cells. In some embodiments, the at least one stem cell type is mesenchymal stem cells and the at least one adult stem cell marker is keratin 14 (K14). In a representative embodiment, the at least one adult stem cell type is hematopoietic stem cells and the at least one adult stem cell marker is selected from tyrosine kinase with immunoglobulin-like and EGF-like domains 1 (TIE1) and CD48.

In some embodiments, the methods include isolating mRNA from the test sample and using quantitative real time PCR to quantify the expression of the at least one adult stem cell marker. In some embodiments, the expression of the at least one adult stem cell marker is quantified by hybridization of a nucleic acid obtained from the test sample to an array of nucleic acid probes.

In some embodiments, the amount of the at least one adult stem cell type in the test sample is determined by comparing the expression of the at least one adult stem cell marker in the test sample to a standard curve that relates the quantity of expression of the at least one adult stem cell marker to the proportion of the at least one adult stem cell type in a sample. Methods of creating a standard curve include quantifying the expression of the at least one adult stem cell marker in at least one first control sample containing a known amount if the at least one adult stem cell type; quantifying the expression of the at least one adult stem cell marker in at least one second control sample that does not contain a detectable amount of the at least one adult stem cell type; and correlating the expression of the at least one adult stem cell marker to the amount of the at least one adult stem cell type. In some embodiments, the expression of the at least one adult stem cell marker is quantified in at least ten first and at least ten second control samples.

Also provided are methods for quantifying differentiated cells in a test sample. The methods include quantifying the expression of at least one differentiated cell marker in the test sample; and determining the amount of the at least one differentiated cell type in the test sample by comparing the expression of the at least one differentiated cell marker in the test sample to the expression of the at least one differentiated cell marker in a control sample containing a known amount of the at least one differentiated cell type.

In some embodiments, the at least one differentiated cell type is selected from chondrocytes, osteoblasts, and fibroblasts. In some embodiments, the at least one differentiated cell type marker is selected from stanniocalcin 1 (STC1), bone morphogenetic protein 10 (BMP10), and E-cadherin. In some embodiments, the at least one differentiated cell type is chondrocytes and the at least one differentiated cell type marker is stanniocalcin 1 (STC1). In a representative embodiment, the at least one differentiated cell type is osteoblasts and the at least one differentiated cell type marker is bone morphogenetic protein 10 (BMP10). In yet another embodiment, the at least one differentiated cell type is fibroblasts and the at least one differentiated cell type marker is E-cadherin.

In some embodiments, the methods include isolating mRNA from the test sample and using quantitative real time PCR to quantify the expression of the at least one differentiated cell marker. In some embodiments, the expression of the at least one differentiated cell marker is quantified by hybridization of a nucleic acid obtained from the test sample to an array of nucleic acid probes.

In some embodiments, the amount of the at least one differentiated cell type in the test sample is determined by comparing the expression of the at least one differentiated cell marker in the test sample to a standard curve that relates the quantity of expression of the at least one differentiated cell marker to the proportion of the at least one differentiated cell type in a sample. The standard curve may be created by a method comprising the steps of: quantifying the expression of the at least one differentiated cell marker in at least one first control sample containing a known amount of the at least one differentiated cell type; quantifying the expression of the at least one differentiated cell marker in at least one second control sample that does not contain a detectable amount of the at least one differentiated cell type; and correlating the expression of the at least one differentiated cell marker to the amount of the at least one differentiated cell type. In some embodiments, the expression of the at least one differentiated cell marker is quantified in at least ten first and at least second control samples.

Also provided are kits that include a container comprising at least one first control sample containing a known amount of at least one adult stem cell type; a container comprising at least one second control sample that does not contain a detectable amount of the at least one adult stem cell type; and primers for quantifying at least one adult stem cell marker. Further provided are kits that include a container comprising at least one first control sample containing a known amount of at least one differentiated cell type; a container comprising at least one second control sample that does not contain a detectable amount of the at least one differentiated cell type; and primers for quantifying at least one differentiated cell type. The kits may further include instructions for creating a standard curve. The instructions direct quantifying the expression of the at least one adult stem cell marker from at least one first control sample; quantifying the expression of the at least one adult stem cell marker from at least one second control sample; and correlating the expression of the at least one adult stem cell marker to the amount of the at least one adult stem cell type.

### BRIEF DESCRIPTION OF THE DRAWINGS

FIG. 1 shows standard curve plots of the log of the starting copy number (Log Co) versus threshold cycle (Ct) for real time PCR experiments described in Example 3. Shown are plots for (A) BMP10 in osteoblasts, (B) keratin 14 in mesenchymal stem cells, (C) CD48 in CD34⁺ stem cells, (D) E-cadherin in dermal fibroblasts, (E) TIE1 in CD34⁺ stem cells, and (F) STC1 in chondrocytes.

FIG. 2 shows an overview of a method of quantifying cells of interest in a test sample. The figure shows (A) isolating cells from a tissue known to have adult stem cells and isolating total RNA from that tissue sample and (B) isolating cells from a tissue known to not have adult stem cells and isolating total RNA from that tissue sample. The total RNA from the tissue samples is mixed and reverse transcribed. The figure also shows (C) isolating cells from a tissue containing an unknown pool of cells and isolating total RNA. The figure also shows (D) real time PCR of the cDNA from (A), (B), and (C). The figure further shows (E) generation of a standard curve and analysis of the test sample.

### DETAILED DESCRIPTION OF CERTAIN EMBODIMENTS

The present invention provides a novel and simple method for quantifying the presence of certain cell types by quantifying the expression of certain cell markers. The inventors performed a whole genome interrogation of five different cell types and discovered different expression patterns for each of these cells. These different expression patterns of cell markers can be used to identify the presence of certain cell types in a sample. Furthermore, the cell markers can be used to quantify specific cell types in a sample by comparing the expression of the cell marker in a sample to the expression of the cell marker in a control sample containing a known amount of the cell.

### DEFINITIONS

The section headings used herein are for organizational purposes only and are not to be construed as limiting the subject matter described. All documents, or portions of documents, cited herein, including but not limited to patents, patent applications, articles, books, and treatises, are hereby expressly incorporated by reference in their entirety for any purpose. In the event that one or more of the incorporated documents or portions of documents defines a term that contradicts that term's definition in this application, this application controls.

The word “a” or “an” means “at least one” unless specifically stated otherwise. The use of “or” means “and/or” unless stated otherwise. The use of “or” in the context of multiply dependent claims means the alternative only. The meaning of the phrase “at least one” is equivalent to the meaning of the phrase “one or more.” Furthermore, the use of the term “including,” as well as other forms, such as “includes” and “included,” is not limiting. Also, terms such as “element” or “component” encompass both elements or components comprising one unit and elements or components that comprise more than one unit unless specifically stated otherwise. All ranges discussed herein include the endpoints and all values between the endpoints.

The terms “nucleic acid molecule” and “polynucleotide” may be used interchangeably, and refer to a polymer of nucleotides. Such polymers of nucleotides may contain natural and/or non-natural nucleotides, and include, but are not limited to, DNA, RNA, and PNA. “Nucleic acid sequence” refers to the linear sequence of nucleotides that comprise the nucleic acid molecule or polynucleotide.

The term “primer” refers to a nucleic acid molecule that can bind to a target sequence and can be extended by amplification.

The term “probe” refers to a nucleic acid molecule, polynucleotide, peptide, or protein, that can be recognized by a particular molecule.

The terms “polypeptide” and “protein” are used interchangeably to refer to a polymer of amino acid residues, and are not limited to a minimum length. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like.

As used herein a “stem cell” refers to an undifferentiated cell that has the ability to renew itself for long periods of time by cell division and can give rise to specialized cells that make up the tissues and organs of the body. The process by which an undifferentiated cell acquires the features of a specialized cell is called “differentiation.” The ability of stem cells from one tissue to differentiate into cells of another tissue is referred to as “plasticity” or “transdifferentiation.”

Stem cells can be totipotent, pluripotent, or multipotent. “Totipotent” stem cells have the ability to develop into any cell type, including extra-embryonic tissues. “Pluripotent” stem cells have the ability to give rise to all types of cells that develop from the three germ layers, i.e., mesoderm, endoderm, and ectoderm. Experimentally, pluripotency can be determined by the formation of a teratoma that includes cells from all three germ layers, after injection of a pluripotent stem cell into a test animal. Pluripotent stem cells, however, cannot give rise to extra-embryonic tissues such as the amnion, chorion, and other components of the placenta. “Multipotent” stem cells can differentiate into a limited number of cell types.

An “embryonic stem cell” refers to an undifferentiated pluripotent stem cell that is derived from the inner cell mass of a blastocyst.

An “adult stem cell” refers to an undifferentiated cell found in a tissue that can renew itself and differentiate to give rise to all the specialized cell types of the tissue from which it originated, and possibly other tissues. Representative adult stem cells include, for example and without limitation, mesenchymal stem cells, hematopoietic stem cells, adipocytic stem cells, pulmonary epithelial stem cells, gastrointestinal tract stem cells, pancreatic stem cells, hepatic oval stem cells, mammary and prostatic gland stem cells, ovarian and testicular stem cells, bone marrow stem cells, stromal stem cells, cardiac stem cells, neural stem cells, skin stem cells, fibrocytes, and ocular stem cells.

A “mesenchymal stem cell (MSC)” refers to a type of adult stem cell that has the capacity to self-renew and differentiate into various cell types. Mesenchymal stem cells are also known as marrow stromal stem cells, stromal precursor cells, mesenchymal progenitor cells, and colony-forming unit-fibroblastic (CFU-F) cells. Traditionally, mesenchymal stem cells were thought to be multipotent and have the capability to differentiate along mesenchymal lineages, e.g., bone, cartilage, ligament, tendon, stroma, adipose, and muscle. See e.g., Bobis et al., Folia Histochem Cytobiol. (2006) 44(4):215-230, Bajada et al., J. Tissue Eng. Regen. Med. (2008) 2(4):169-183, Pittenger et al., Science (1999) 284(5411):143-147. However, some studies indicate that MSCs may, under certain conditions, have the capability to differentiate along endodermal and ectodermal lineages. See e.g., Bobis et al., Folia Histochem Cytobiol. (2006) 44(4):215-230, Bajada et al., J. Tissue Eng. Regen. Med. (2008) 2(4):169-183, Chamberlain et al., Stem Cells (2007) 25(11):2739-2749, Sasaki et al., J Immunol. (2008) 180(4):2581-2587. Thus, mesenchymal stem cells may differentiate into multiple cells types including, but not limited to, adipocytes, chondrocytes, osteoblasts, cells of the visceral mesoderm (endothelial cells), neurons, neural cells, glia, brain astrocytes, endoderm cells, fibroblasts, myoblasts, hepatocytes, tenocytes, cardiomyocytes, pancreatic β cells, cenocytes, skeletal myocytes, keratinocytes, and pericytes. See, e.g., Bobis et al., Folia Histochem Cytobiol. (2006) 44(4):215-230, Dazzi et al., Blood Rev. (2005) 20(3):161-171.

In some embodiments, mesenchymal stem cells may be present in adipose tissue, amniotic fluid, bone, bone marrow, chorionic villi of the placenta, exfoliated deciduous teeth, fetal tissue, fetal liver, fetal lung, hair follicles, liver, lung, myocardium, periosteum, peripheral blood, placental blood, skeletal muscle, skin, spleen, synovia, synovial fluid, tendon, umbilical cord blood, and umbilical cord tissue from mammals.

“Mammals” include, but are not limited to, rodents, simians, humans, felines, canines, equines, bovines, porcines, ovines, caprines, mammalian laboratory animals, mammalian farm animals, mammalian sport animals, and mammalian pets.

A “hematopoietic stem cell (CD34⁺ stem cell or HSC)” is a type of adult stem cell that has the capacity to self-renew and differentiate into all circulating blood cells in an adult. Hematopoietic stem cells express CD34 on the cell surface. Hematopoietic stem cells may differentiate into erythrocytes, platelets, basophils, eosinophils, neutrophils, granulocytes, dendritic cells, monocytes, macrophages, B cells, T cells, and NK cells.

In some embodiments, hematopoietic stem cells are present in bone marrow, umbilical cord blood, placenta, peripheral blood, fetal liver, fetal spleen, and aorta-gonad-mesonephros.

A “differentiated cell” refers to a cell with a specialized function. Exemplary differentiated cells include, but are not limited to, adipocytes, chondrocytes, osteoblasts, cells of the visceral mesoderm (endothelial cells), neurons, neural cells, glia, brain astrocytes, endoderm cells, fibroblasts, myoblasts, hepatocytes, tenocytes, cardiomyocytes, pancreatic β cells, cenocytes, skeletal myocytes, keratinocytes, erythrocytes, platelets, basophils, eosinophils, neutrophils, granulocytes, dendritic cells, monocytes, macrophages, B cells, T cells, NK cells, kidney cells, and colon cells.

As used herein, a “test sample” includes fluids such as blood, serum, plasma, urine, cerebrospinal fluid, tears, saliva, lymph, dialysis fluid, lavage fluid, semen, and other liquid samples or tissues of biological origin. It includes cells or cells derived therefrom and the progeny thereof, including cells in culture, cell supernatants, and cell lysates. It includes organ or tissue culture derived fluids, tissue biopsy samples, tumor biopsy samples, stool samples, and fluids extracted from physiological tissues. Cells dissociated from solid tissues, tissue sections, and cell lysates are included. The definition also includes samples that have been manipulated in any way after their procurement, such as by treatment with reagents, solubilization, or enrichment for certain components, such as polynucleotides or polypeptides. Also included in the term are derivatives and fractions of biological samples. A test sample can be used in methods for quantifying adult stem cells. A test sample can also be used in methods for quantifying differentiated cells.

In some embodiments, a test sample is obtained from a tissue selected from adipose tissue, amniotic fluid, bone, aorta-gonad-mesonephros, bone marrow, chorionic villi of the placenta, exfoliated deciduous teeth, fetal tissue, fetal liver, fetal lung, fetal spleen, hair follicles, liver, lung, myocardium, periosteum, peripheral blood, placenta, placental blood, skeletal muscle, skin, spleen, synovia, synovial fluid, tendon, umbilical cord blood, and umbilical cord tissue. In other embodiments, a test sample is selected from skin, bone, cartilage, peripheral blood, bone marrow, and spleen.

The term “patient” refers to a mammal. In some embodiments, a patient has been administered at least one adult stem cell. In some embodiments, a patient has at least one of acute myelogenous leukemia, acute lymphoblastic leukemia, chronic lymphocytic leukemia, chronic myelogenous leukemia, multiple myeloma, non-Hodgkin lymphomas, diffuse large B cell lymphoma, mantel cell lymphoma, Hodgkin lymphoma, aplastic anemia, testicular germ-cell cancer, sickle cell disease, thalassemia, systemic lupus erythematosus, chronic skin wounds, osteogenesis imperfecta, cartilage lesions, myocardial infarction, stroke, traumatic brain injury, kidney disease, lung disease, muscle damage, Parkinson's disease, multiple sclerosis, and psoriasis.

The term “isolating” as used herein refers to separating a molecule from at least some of the components with which it is typically found in nature. For example, a polypeptide is referred to as “isolated” when it is separated from at least some of the components of the cell in which it was produced. Where a polypeptide is secreted by a cell after expression, physically separating the supernatant containing the polypeptide from the cell that produced it is considered to be “isolating” the polypeptide. Similarly, a polynucleotide is referred to as “isolated” when it is not part of the larger polynucleotide (such as, for example, genomic DNA or mitochondrial DNA, in the case of a DNA polynucleotide) in which it is typically found in nature, or is separated from at least some of the components of the cell in which it was produced, e.g., in the case of an RNA polynucleotide.

A cell “marker” refers to a gene or polypeptide whose expression level, either alone or in combination with expression of other genes or polypeptides, distinguishes one type of cell from at least a plurality of other types of cells. In some embodiments the marker distinguishes the cell type from all other cell types. The expression level of a marker may be at least about one and a half-fold, at least about two-fold, at least about three-fold, at least about five-fold, at least about ten-fold, at least about 20-fold, at least about 30-fold, at least about 50-fold, at least about 100-fold, at least about 200-fold, at least about 300-fold, at least about 400-fold, at least about 500-fold, or at least about 1000-fold greater in the cell of interest compared to other cells. A cell marker may include an adult stem cell marker or a differentiated cell marker.

An “adult stem cell marker” refers to a gene or polypeptide whose expression level, either alone or in combination with expression of other genes or polypeptides, distinguishes adult stem cells from other types of stem cells and from differentiated cells. The expression level of an adult stem cell marker may be at least about one and a half-fold, at least about two-fold, at least about three-fold, at least about five-fold, at least about ten-fold, at least about 20-fold, at least about 30-fold, at least about 50-fold, at least about 100-fold, at least about 200-fold, at least about 300-fold, at least about 400-fold, at least about 500-fold, or at least about 1000-fold greater in the adult stem cell of interest compared to other cells. Exemplary stem cell markers include, but are not limited to, tyrosine kinase with immunoglobulin-like and EGF-like domains 1 (TIE1), CD48, and kertain 14 (K14). A sample containing adult stem cells may be obtained from tissue selected from adipose tissue, amniotic fluid, aorta-gonad-mesonephros, bone, bone marrow, chorionic villi of the placenta, exfoliated deciduous teeth, fetal tissue, fetal liver, fetal lung, fetal spleen, hair follicles, liver, lung, myocardium, periosteum, peripheral blood, placenta, placental blood, skeletal muscle, skin, spleen, synovia, synovial fluid, tendon, umbilical cord blood, and umbilical cord tissue.

As used herein, “keratin 14 (K14)” refers to all mammalian forms of the protein including, for example, alternative splice isoforms and naturally occurring isoforms. Representative K14 species include, without limitation, human (GenBank Accession No. NM_{—}000526), rat (GenBank Accession No. NM_{—}001008751), and mouse (GenBank Accession No. NM_{—}016958) forms. K14 is an adult stem cell marker for mesenchymal stem cells. K14 expression may be at least about one and a half-fold, at least two-fold, at least about three-fold, at least about five-fold, at least about ten-fold, at least about 20-fold, at least about 30-fold, at least about 50-fold, at least about 100-fold, at least about 200-fold, at least about 300-fold, at least about 400-fold, at least about 500-fold, or at least about 1000-fold greater in mesenchymal stem cells compared to other cells.

As used herein, “tyrosine kinase with immunoglobulin-like and EGF-like domains (TIE1)” refers to all mammalian forms of the protein including, for example, alternative splice isoforms and naturally occurring isoforms. Representative TIE1 species include, without limitation, human (GenBank Accession No. NM_{—}005424), rat (GenBank Accession No. NM_{—}001105737), and mouse (GenBank Accession No. NM_{—}011587) forms. TIE1 is an adult stem cell marker for CD34⁺ stem cells. TIE1 expression may be at least about one and a half-fold, at least two-fold, at least about three-fold, at least about five-fold, at least about ten-fold, at least about 20-fold, at least about 30-fold, at least about 50-fold, at least about 100-fold, at least about 200-fold, at least about 300-fold, at least about 400-fold, at least about 500-fold, or at least about 1000-fold greater in hematopoietic stem cells compared to other cells.

As used herein, “CD48” refers to all mammalian forms of the protein including, for example, alternative splice isoforms and naturally occurring isoforms. Representative CD48 species include, without limitation, human (GenBank Accession No. NM_{—}001778), rat (GenBank Accession No. NM_{—}139103), and mouse (GenBank Accession No. NM_{—}007649) forms. CD48 is also an adult stem cell marker for CD34⁺ stem cells. CD48 expression may be at least about one and a half-fold, at least two-fold, at least about three-fold, at least about five-fold, at least about ten-fold, at least about 20-fold, at least about 30-fold, at least about 50-fold, at least about 100-fold, at least about 200-fold, at least about 300-fold, at least about 400-fold, at least about 500-fold, or at least about 1000-fold greater in hematopoietic stem cells compared to other cells.

Hematopoietic stem cells may be defined by expression of the marker CD34. Thus, as used herein, “CD34” refers to an adult stem cell marker for hematopoietic stem cells, and includes all mammalian forms of the protein including, for example, alternative splice isoforms and naturally occurring isoforms. Representative CD34 species include, without limitation, human (GenBank Accession No. NM_{—}001025109 and GenBank Accession No. NM_{—}001773), rat (GenBank Accession No. NM_{—}001107202), and mouse GenBank Accession No. NM_{—}001111059 and GenBank Accession No. NM_{—}133654) forms.

A “differentiated cell marker” refers to a gene or polypeptide whose expression level, either alone or in combination with expression of other genes or polypeptides, distinguishes a differentiated cell from undifferentiated cells, which include, for example, adult stem cells. The expression level of a differentiated cell marker may be at least about one and a half-fold, at least about two-fold, at least about three-fold, at least about five-fold, at least about ten-fold, at least about 20-fold, at least about 30-fold, at least about 50-fold, at least about 100-fold, at least about 200-fold, at least about 300-fold, at least about 400-fold, at least about 500-fold, or at least about 1000-fold greater in the differentiated cell of interest compared to other cells. Exemplary differentiated cell markers include, but are not limited to, bone morphogenic protein 10 (BMP-10), stanniocalcin 1 (STC1), and E-cadherin. A sample containing differentiated cells may be obtained from tissue selected from skin, bone, cartilage, peripheral blood, bone marrow, and spleen, for example.

As used herein “bone morphogenetic protein 10 (BMP10)” refers to all mammalian forms of the protein including, for example, alternative splice isoforms and naturally occurring isoforms. Representative BMP10 species include, without limitation, human (GenBank Accession No. NM_{—}014482), rat (GenBank Accession No. NM_{—}001031824), and mouse (GenBank Accession No. NM_{—}009756) forms. In some embodiments, BMP10 is a marker for osteoblasts. In some embodiments, BMP10 gene expression is at least about one and a half-fold, at least about two-fold, at least about three-fold, at least about five-fold, at least about ten-fold, at least about 20-fold, at least about 30-fold, at least about 50-fold, at least about 100-fold, at least about 200-fold, at least about 300-fold, at least about 400-fold, at least about 500-fold, or at least about 1000-fold greater in osteoblasts compared to other cells.

As used herein, “stanniocalcin 1 (STC1)” refers to all mammalian forms of the protein including, for example, alternative splice isoforms and naturally occurring isoforms. Representative STC1 species include, without limitation, human (GenBank Accession No. NM_{—}003155), rat (GenBank Accession No. NM_{—}031123), and mouse (GenBank Accession No. NM_{—}009285) forms. In some embodiments, STC1 is a marker for chondrocytes. In some embodiments, STC1 expression is at least about one and a half-fold, at least about two-fold, at least about three-fold, at least about five-fold, at least about ten-fold, at least about 20-fold, at least about 30-fold, at least about 50-fold, at least about 100-fold, at least about 200-fold, at least about 300-fold, at least about 400-fold, at least about 500-fold, or at least about 1000-fold greater in chondrocytes compared to other cells.

As used herein, “E-cadherin” refers to all mammalian forms of the protein including, for example, alternative splice isoforms and naturally occurring isoforms. Representative E-cadherin species include, without limitation, human (GenBank Accession No. NM_{—}004360), rat (GenBank Accession No. NM_{—}031334), and mouse (GenBank Accession No. NM_{—}009864) forms. In some embodiments, E-cadherin is a marker for fibroblasts. In some embodiments, E-cadherin expression is at least about one and a half-fold, at least about two-fold, at least about three-fold, at least about five-fold, at least about ten-fold, at least about 20-fold, at least about 30-fold, at least about 50-fold, at least about 100-fold, at least about 200-fold, at least about 300-fold, at least about 400-fold, at least about 500-fold, or at least about 1000-fold greater in fibroblasts compared to other cells.

### Methods of Quantifying the Expression of a Cell Marker of Interest

The expression of a cell marker of interest may be quantified in a number of ways known in the art. Exemplary methods include, but are not limited to, hybridization-based assays and amplification-based assays.

The hybridization-based assay includes hybridizing a nucleic acid obtained from a test sample to an array of nucleic acid probes. In some embodiments, the hybridization-based assay uses microarray technology as is known in the art (see, e.g., Pollack et al., Nature Genetics (1999) 23: 41-46; Pastinen, Genome Res. (1997) 7: 606-614; Jackson, Nature Biotechnology (1996) 14: 1685; Chee, Science (1995) 274: 610; and Pinkel et al. Nature Genetics (1998) 20:207-211).

Microarrays comprise one or more molecules that specifically bind to a cell marker of interest. For example, nucleic acids, such as RNA, are isolated from a sample and the isolated nucleic acids are contacted with one or more nucleic acids in the microarray to form a hybridization complex. The amount of the hybridization complex may be quantified by binding of a labeled molecule to a hybridization complex and quantitatively detecting the label.

Alternatively, an amplification-based assay may be used to determine the expression of a cell marker of interest. The amplification-based assay includes quantitative real time PCR (see, e.g., Poropat et al. Clinical Chem. (1998) 44:724-730). In such embodiments, RNA is isolated from one or more samples and is reverse transcribed. The resulting DNA is then amplified using real time PCR.

The expression of a cell marker of interest may also be analyzed using a classification system as is known in the art. For example, a cell marker of interest may be analyzed using the PANTHER™ Classification System (see, e.g., Thomas et al. Genome Research (2003) 13:2129-2141).

### Methods of Determining the Amount of a Cell Type of Interest in a Test Sample

The amount of a cell type of interest may be determined in a number of ways known in the art. The amount of the at least one adult stem cell type in the test sample includes comparing the expression of the at least one adult stem cell marker in the test sample to the expression of the at least one adult stem cell marker in a control sample containing a known amount of the at least one adult stem cell type. In certain embodiments, a control sample is created by isolating cells from a tissue or a cell line. A control sample may also be created by mixing together a known amount of a cell type of interest and a known amount of cells that do not include the cell type of interest.

Exemplary methods include, but are not limited to, comparing the expression of the at least one cell marker of interest in the test sample to a standard curve that relates the quantity of expression of the at least one cell marker of interest to the proportion of the at least one cell type of interest in a sample.

A standard curve may be generated by creating at least one first control sample containing a known amount of the at least one cell type of interest; creating at least one second control sample that does not contain a detectable amount of the at least one cell type of interest; quantifying the expression of the at least one cell marker of interest in the at least one first control sample; quantifying the expression of the at least one cell marker of interest in the at least one second control sample; and correlating the expression of the at least one cell marker of interest to the amount of the at least one cell type of interest. In some embodiments, at least ten first control samples and at least ten second control samples are created, and the expression of the at least one cell marker of interest is quantified in each of the at least ten first and second control samples. Samples can be generated by serially diluting known amounts of RNA from a cell type of interest into a background of known amounts of RNA from other cell types. The expression of a cell marker of interest can then be determined from each of the samples, using a standard curve relating the expression of a cell marker of interest and the amount of the cell type of interest.

The expression of the at least one cell marker of interest may be correlated to the amount of the at least one adult stem cell type by determining the number of copies of the at least one cell marker of interest per the number of cells of interest as is known in the art. Qualitative real time PCR may be used to determine the number of copies of the at least one cell marker of interest per the number of cell types of interest in a control sample. Determining the number of copies of the at least one cell marker of interest per the number of cell types of interest may include performing limiting dilution of a sample. To perform limiting dilution analysis, a known amount of a cell type of interest may be serially diluted into a background of control cells, and total RNA can be obtained from those cells. After reverse transcription, the resulting cDNA may be subjected to real time PCR. The number of copies of the at least one cell markers of interest per the number of cell types of interest may then be calculated. In certain such embodiments, the number of copies of the at least one cell marker of interest per the number of cell types of interest in a control sample is compared to the number of copies of the at least one adult stem cell marker in a test sample and the number of adult stem cell types is calculated.

A control sample may be obtained from tissue known to have low numbers of adult stem cells. Exemplary tissues include kidney and colon. A control sample that does not contain a detectable amount of the at least one adult stem cell type may be obtained from cells such as adipocytes, brain astrocytes, cardiomyocytes, cenocytes, chondrocytes, cells of the visceral mesoderm (endothelial cells), endoderm cells, fibroblasts, glial cells, hepatocytes, keratinocytes, myoblasts, neural cells, neurons, osteoblasts, pancreatic β cells, skeletal myocytes, tenocytes, erythrocytes, platelets, basophils, eosinophils, neutrophils, granulocytes, dendritic cells, monocytes, macrophages, B cells, T cells, and NK cells.

A control sample containing a known amount of at least one differentiated cell type may include chondrocytes, osteoblasts, and fibroblasts. A control sample that does not contain a detectable amount of at least one differentiated cell type may include mesenchymal stem cells and hematopoietic stem cells.

### Kits

Also provided are kits for quantifying a cell type of interest. The kits may include a container comprising a control sample containing a known amount of a cell of interest and a container comprising a control sample that does not contain a detectable amount of a cell of interest. The kits may alternatively include a container comprising a control sample containing a known amount of RNA isolated from a cell of interest and a container comprising a control sample containing RNA isolated from a cell other than the cell of interest. The kits may also include primers for quantifying at least one cell marker of interest. For example, the primers may be designed for amplifying adult stem cell markers such as TIE1, CD48, K14, and CD34. The primers may also be useful for amplifying differentiated cell markers such as STC1, BMP-10, and E-cadherin. Primers may be obtained from TaqMan Gene Expression Assay kits. The kits may further include written instructions for creating a standard curve. Exemplary instructions for creating a standard curve include quantifying the expression of the at least one adult stem cell marker from the at least one first control sample; quantifying the expression of the at least one adult stem cell marker from the at least one second control sample; and correlating the expression of the at least one adult stem cell marker to the amount of the at least one adult stem cell type.

### EXAMPLES

The examples discussed below are intended to be purely exemplary and should not be considered to be limiting in any way. The examples are not intended to represent that the experiments below are all or the only experiments performed.

### Example 1

### RNA Isolation

Cells

Primary cells, including mesenchymal stem cells (Lonza, Walkersville, Md., PT-2501), CD34⁺ stem cells (Lonza, 2M-101C), chondrocytes (Lonza, CC-2550), dermal fibroblasts (Cascade Biologics, Carlsbad, Calif., C-013-5C), and osteoblasts (Lonza, CC-2538) were obtained from the indicated vendors. All cells were frozen when received from the manufacturer and stored in liquid nitrogen. Mesenchymal stem cells were thawed and cultured for two passages in Mesenchymal Stem Cell Basal Medium with Growth Supplement (Lonza, PT-3001) according to the manufacturer's protocol for Poietics® Human Mesenchymal Stem Cells. Dermal fibroblasts were thawed and cultured for three passages in Medium 106 (Cascade Biologics, M-106-500) supplemented with Low Serum Growth Supplement (Cascade Biologics, S-003-10). CD34⁺ stem cells, chondrocytes, and osteoblasts were not cultured.

RNA Isolation

Total RNA was isolated from 5×10⁶ mesenchymal stem cells, 1×10⁶CD34⁺ stem cells, 7.5×10⁵ chondrocytes, 3.8×10⁶ dermal fibroblasts, and 1.5×10⁶ osteoblasts using TRIzol® (Invitrogen, Carlsbad, Calif.). Mesenchymal stem cells and dermal fibroblasts were collected, spun down, washed with fresh, warmed media, and resuspended in 1 ml of TRIzol®. CD34⁺ stem cells, chondrocytes, and osteoblasts were thawed, washed with fresh, warmed media, spun down, and resuspended in 1 ml TRIZOL®. Cells were incubated at room temperature for five minutes. 200 ml of chloroform was added and the cells were shook for 15 seconds followed by incubation at room temperature for three minutes. The aqueous phase was transferred to a new tube and mixed with 500 μl of 70% ethanol. That mixture was then added to RNeasy Mini Spin columns (Qiagen, Valencia, Calif.) with on-column DNaseI digestion according to the manufacturer's protocol. RNA quantity and quality were assessed using an Experion Automated Electrophoresis System and its corresponding software (Bio-Rad, Hercules, Calif.) along with the Agilent 2100 Bioanalyzer (Agilent Technologies, Santa Clara, Calif.) according to the manufacture's instructions. This procedure yielded RNA integrity numbers (RINs) that ranged between 9.7 and 10. The RNA samples were aliquoted and stored at −80° C. until the time of use.

### Example 2

### Microarray Analysis

Microarray analysis was performed on the above RNA samples to identify representative genes from MSCs, CD34⁺ stem cells, chondrocytes, dermal fibroblasts, and osteoblasts. Gene expression profiles were generated using the Applied Biosystems Human Genome Survey Microarray, which uses 32,878 60-mer oligonucleotide probes to interrogate 29,098 genes (Applied Biosystems, Foster City, Calif.). The Vanderbilt Microarray Shared Resource labeled the nucleic acid and generated raw microarray data. Briefly, 500 ng RNA was converted to digoxidenin (DIG)-labeled cRNA using the NanoAmp™ RT-IVT Labeling Kit (Applied Biosystems) according to the manufacturer's instructions. Ten μg of labeled cRNA was added to the microarray. Microarray processing, chemiluminescence detection, imaging, auto gridding, and image analysis were performed according to Applied Biosystems's protocols. Raw data were analyzed using the ABarray data analysis package (Applied Biosystems). Probe signal intensities across microarrays were normalized using the quantile method. Features with signal/noise values ≧3 and quality flag values <5,000 were considered detected and were compared by t-test using a fold change ≧1.6, a Benjamini and Hochberg False Discovery Rate (FDR) of <0.05 (19), and/or a p-value of <0.05.

The above microarray analysis detected 16,383 total probes, corresponding to more than 10,000 annotated genes, across all of the cells analyzed (data not shown). To analyze the function of the identified genes, the identified genes were categorized according to the PANTHER™ Classification System. Gene expression data from all of the identified genes was then entered into a Microsoft Access database to further investigate relative expression levels.

To compare relative expression levels, greater than a 1000-fold change in gene expression in one cell type relative to the other cell types analyzed was considered a high-fold change while less than a 500-fold change in one cell type relative to the other cell types analyzed was considered a low-fold change. Genes were categorized into the following groups: (1) a high-fold change in CD34⁺ stem cells and MSCs, together, and a low-fold change in chondrocytes, dermal fibroblasts, and osteoblasts; (2) a low-fold change in CD34⁺ stem cells and MSCs, together, and a high-fold change in chondrocytes, dermal fibroblasts, and osteoblasts; (3) a high-fold change in MSCs and a low-fold change in CD34⁺ stem cells, chondrocytes, dermal fibroblasts, and osteoblasts; and (4) a high-fold change in CD34⁺ stem cells and a low-fold change in MSCs, chondrocytes, dermal fibroblasts, and osteoblasts. These categories were used to narrow the list of more than 10,000 identified genes down to 158 genes. 21 genes from the list of 158 genes were selected for further investigation.

Data from the 21 genes is shown in Table 1. Table 1 shows microarray data (described in Experiment 2) and real time PCR data (described in Experiment 3) for the 21 indicated genes in CD34⁺ stem cells (CD34⁺ cells), chondrocytes (Cdrocytes), fibroblasts (Fibro), human mesenchymal stem cells (hMSC), and osteoblasts (Osteo). Columns labeled with a “q” show real time PCR data while columns without a “q” show microarray data. For example, the CD34+ q column shows real time PCR data while the CD34⁺ column shows microarray data. Real time PCR data is shown by the threshold cycle number. Microarray data is shown by the gene expression values. Data regarding of genes of interest in a particular cell type are highlighted gray.

TABLE 1

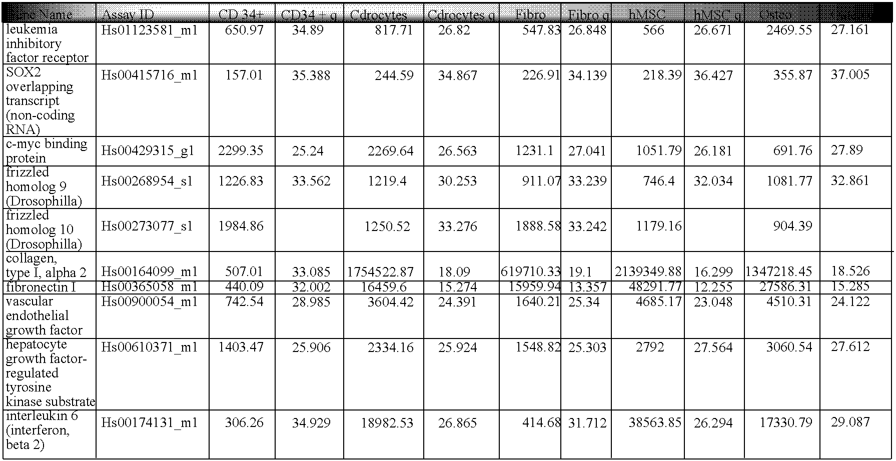

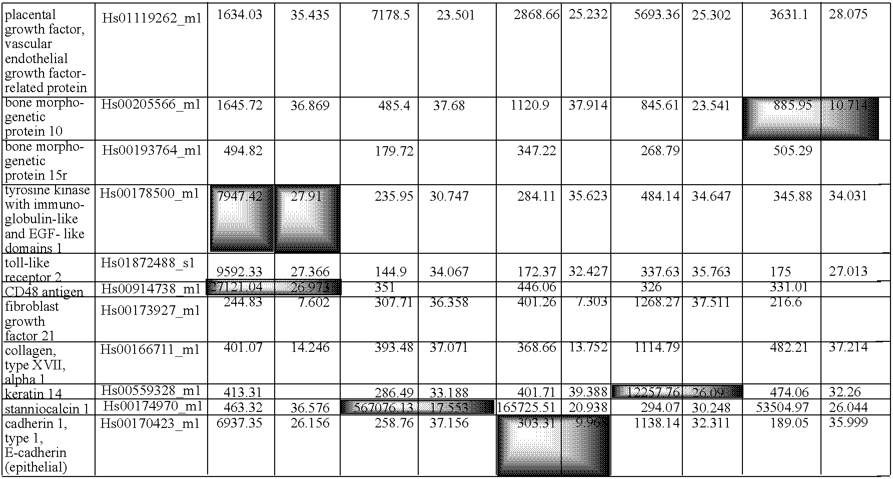

### Example 3

### Real Time PCR Analysis

The microarray data was validated using quantitative real time PCR using a 7500 Fast Real-Time PCR System (Applied Biosystems). cDNA from CD34⁺ stem cells, chondrocytes, dermal fibroblasts, mesenchymal stem cells, and osteoblasts was obtained by reverse transcribing total RNA, which was obtained as described above, using the High Capacity cDNA Reverse Transcription Kit (Applied Biosystems) according to the manufacturer's instructions. The final concentration of cDNA was about 50 ng/μl.

The cDNA was then subjected to real time PCR as follows. FirstChoice® Human Colon Total RNA (Ambion, Austin, Tex., AM7986) was reverse transcribed using the High Capacity cDNA Reverse Transcription Kit (Applied Biosystems) according to the manufacturer's instructions. The final concentration of the colon cDNA was about 50 ng/μl. Real time PCR was performed using TaqMan Gene Expression Assays for the genes of interest. Samples were prepared by mixing together known amounts of cDNA from the colon cDNA sample and the cell of interest cDNA sample as shown in Table 2.

TABLE 2

| % Colon | μl of colon | μl of stem cell |
|---|---|---|
| [cDNA] | [cDNA] | [cDNA] |
| 100 | 10 | 0 |
| 90 | 9 | 1 |
| 80 | 8 | 2 |
| 70 | 7 | 3 |
| 60 | 6 | 4 |
| 50 | 5 | 5 |
| 40 | 4 | 6 |
| 30 | 3 | 7 |
| 20 | 2 | 8 |
| 10 | 1 | 9 |
| 0 | 0 | 10 |

Detection of 18S RNA (Applied Biosystems, 4319413E) in the ligation mixture was used as an endogenous control. The genes of interest were all detected using FAM, while the 18S was detected using VIC. Each reaction was done in triplicate along with a no-template-negative control in 96-well plates. The absolute target quantity was determined using SDS Software v. 1.3.1 (Applied Biosystems) by generating a standard curve where the x-axis provides the log starting copy number (Log Co) and the y-axis provides the threshold cycle (Ct).

Results from the real-time PCR assay are as follows. Table 1 shows the average threshold cycle (Ct) for each target gene in the columns labeled “CD34+ q,” “Cdrocytes q,” “Fibro q,” “hMSC q,” and “Osteo q” for CD34⁺ stem cells, chondrocytes, dermal fibroblasts, mesenchymal stem cells, and osteoblasts, respectively. Genes that had a lower Ct value in one cell type compared to the other four cell types were identified.

Taking together the results of the real-time PCR assay and the microarray assay, the list of 21 genes was narrowed down to six genes of interest. Table 1 shows the microarray data and real-time PCR data for the genes of interest and the corresponding cell type of interest shaded in gray.

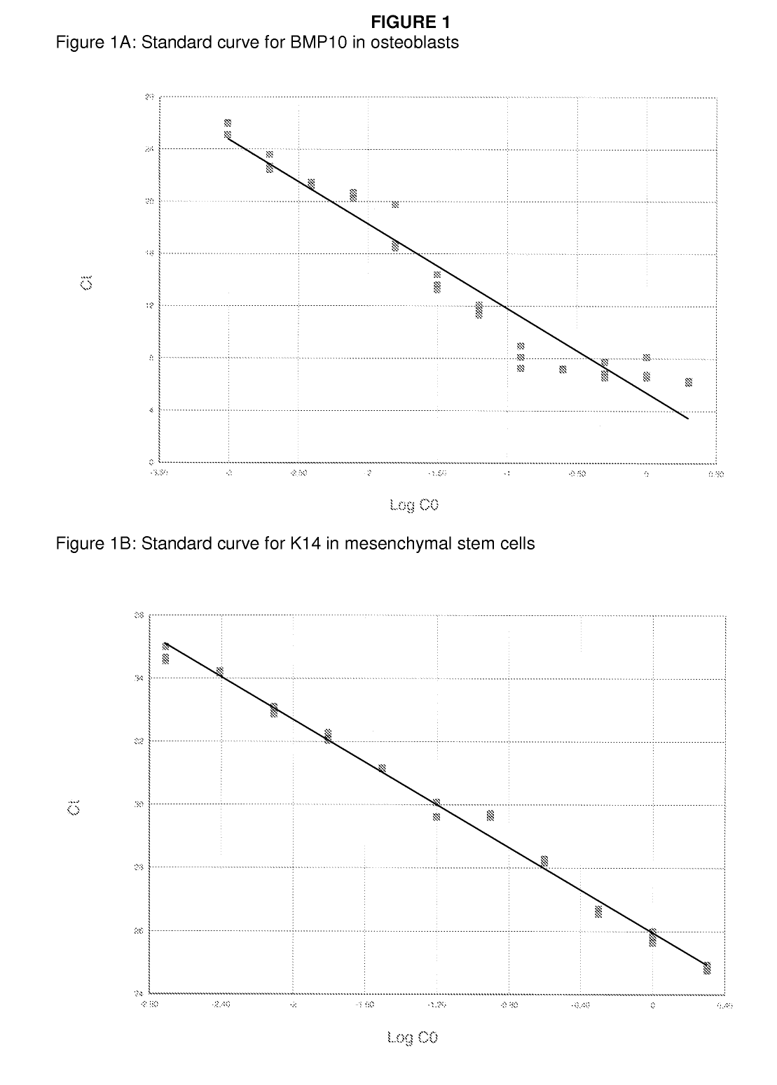

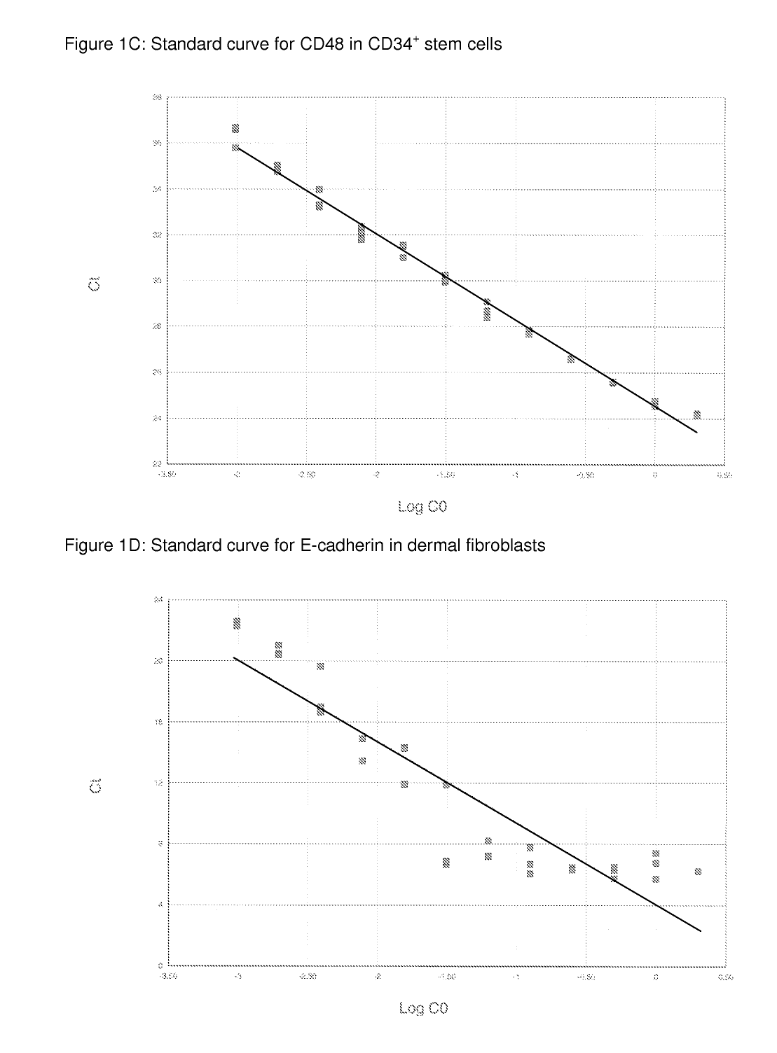

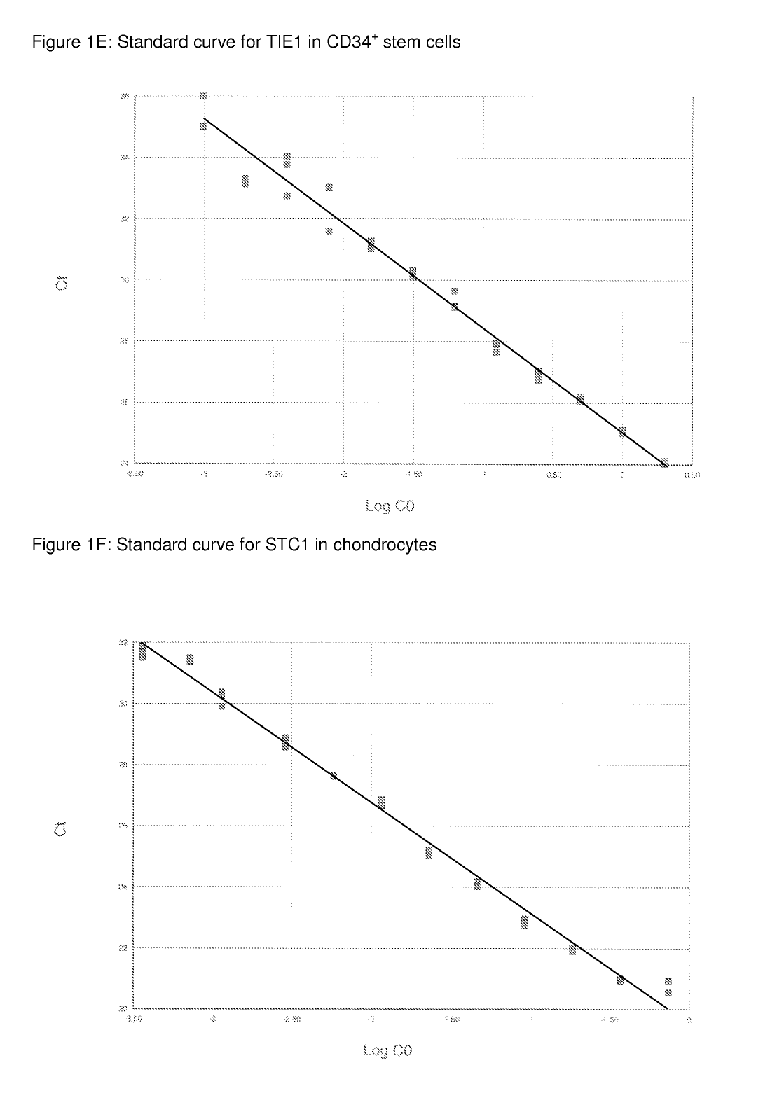

FIG. 1 shows the standard curves for the six genes of interest in particular cell types. Shown are standard curves for the following genes and cell types: (A) BMP10 in osteoblasts; (B) keratin 14 in MSCs; (C) CD48 in CD34⁺ stem cells; (D) E-cadherin in fibroblasts; (E) TIE1 in CD34⁺ stem cells; and (F) STC-1 in chondrocytes. The standard curves show linearity as demonstrated by the least squares fit correlation coefficient (R²) for each standard curve as shown in Table 3.

TABLE 3

| Standard Curve | R² value |
|---|---|
| BMP10 in osteoblasts | 0.935532 |
| keratin 14 in MSCs | 0.990928 |
| CD48 in CD34⁺ stem cells | 0.990244 |
| E-cadherin in fibroblasts | 0.839785 |
| TIE1 in CD34⁺ stem cells | 0.984007 |
| STC-1 in chondrocytes | 0.990501 |

### Example 4

### Determining the Percentage of Stem Cells in a Sample

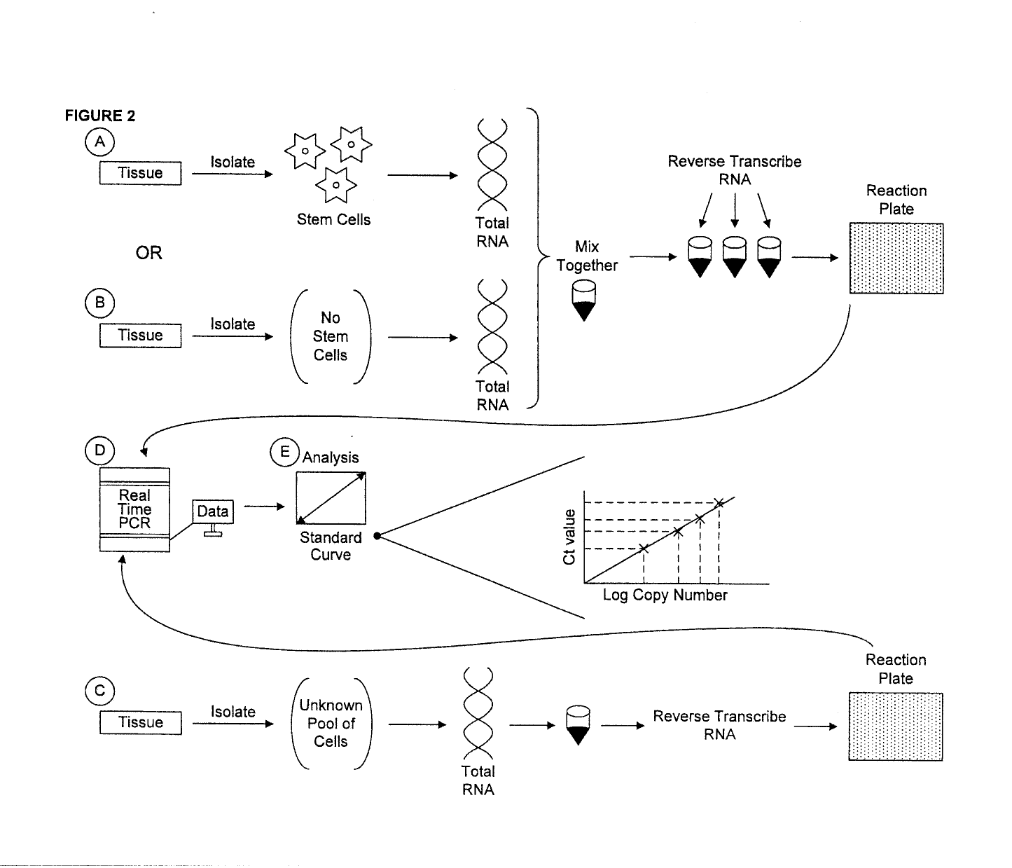

FIG. 2 shows an overview of a method used for quantifying adult stem cells in a sample. First, cells from a tissue known to have adult stem cells are obtained and total RNA from that tissue sample is isolated. Cells from a tissue known to lack adult stem cells are also obtained and total RNA from that tissue sample is also isolated. The total RNA from the tissue samples is mixed in known amounts and reverse transcribed. Real time PCR is performed on the resulting cDNA and a standard curve is generated that correlates the expression of a cell marker to the percentage of the adult stem cells in a sample. Cells from a tissue containing an unknown pool of cells is further obtained and their total RNA is isolated and reverse transcribed. Real time PCR is again performed on the resulting cDNA for an adult stem cell marker and the percentage of adult stem cells present in the sample is determined by comparing the expression of the adult stem cell marker to the standard curve. In this Example, real time PCR is performed as described in Example 3.

It will be readily apparent to one of ordinary skill in the relevant arts that other suitable modifications and adaptations to the methods and applications described herein are suitable and may be made without departing from the scope of the invention or any embodiment thereof. While the invention has been described in connection with certain embodiments, it is not intended to limit the invention to the particular forms set forth, but on the contrary, it is intended to cover such alternatives, modifications and equivalents as may be included within the spirit and scope of the invention as defined by the following claims.

1. A method for quantifying adult stem cells in a test sample, comprising:
quantifying the expression of at least one adult stem cell marker in the test sample; and
determining the amount of the at least one adult stem cell type in the test sample by comparing the expression of the at least one adult stem cell marker in the test sample to the expression of the at least one adult stem cell marker in a control sample containing a known amount of the at least one adult stem cell type.

2. The method of claim 1, wherein the at least one adult stem cell type is selected from mesenchymal stem cells and hematopoietic stem cells.

3. The method of claim 1, wherein the at least one adult stem cell type is mesenchymal stem cells and the at least one adult stem cell marker is keratin 14 (K14).

4. The method of claim 1, wherein the at least one adult stem cell type is hematopoietic stem cells and the at least one adult stem cell marker is selected from tyrosine kinase with immunoglobulin-like and EGF-like domains 1 (TIE1) and CD48.

5. The method of claim 1, wherein the test sample is obtained from tissue selected from adipose tissue, amniotic fluid, aorta-gonad-mesonephros, bone, bone marrow, chorionic villi of the placenta, exfoliated deciduous teeth, fetal tissue, fetal liver, fetal lung, fetal spleen, hair follicles, liver, lung, myocardium, periosteum, peripheral blood, placenta, placental blood, skeletal muscle, skin, spleen, synovia, synovial fluid, tendon, umbilical cord blood, umbilical cord tissue.

6. The method of claim 1, wherein the method further comprises isolating mRNA from the test sample.

7. The method of claim 1, wherein the method further comprises using quantitative real time PCR to quantify the expression of the at least one adult stem cell marker.

8. The method of claim 1, wherein the expression of the at least one adult stem cell marker is quantified by hybridization of a nucleic acid obtained from the test sample to an array of nucleic acid probes.

9. The method of claim 1, wherein the amount of the at least one adult stem cell type in the test sample is determined by comparing the expression of the at least one adult stem cell marker in the test sample to a standard curve that relates the quantity of expression of the at least one adult stem cell marker to the proportion of the at least one adult stem cell type in a sample.

10. The method of claim 9, wherein the standard curve is created by a method comprising the steps of:
quantifying the expression of the at least one adult stem cell marker in at least one first control sample containing a known amount of the at least one adult stem cell type;
quantifying the expression of the at least one adult stem cell marker in at least one second control sample that does not contain a detectable amount of the at least one adult stem cell type; and
correlating the expression of the at least one adult stem cell marker to the amount of the at least one adult stem cell type.

11. The method of claim 10, wherein the at least one first control sample is obtained from cells selected from kidney cells and colon cells.

12. The method of claim 10, wherein the at least one second control sample is obtained from cells selected from adipocytes, brain astrocytes, cardiomyocytes, cenocytes, chondrocytes, cells of the visceral mesoderm (endothelial cells), endoderm cells, fibroblasts, glial cells, hepatocytes, keratinocytes, myoblasts, neural cells, neurons, osteoblasts, pancreatic β cells, skeletal myocytes, tenocytes, erythrocytes, platelets, basophils, eosinophils, neutrophils, granulocytes, dendritic cells, monocytes, macrophages, B cells, T cells, and NK cells.

13. The method of claim 10, wherein the expression of the at least one adult stem cell marker is quantified in at least ten first and at least ten second control samples.

14. A method of quantifying adult stem cells in a patient, comprising:
obtaining a test sample from a patient who has been administered at least one adult stem cell type; and
quantifying the expression of at least one adult stem cell marker in the test sample; and
determining the amount of the at least one adult stem cell type in the test sample by comparing the expression of the at least one adult stem cell marker in the test sample to the expression of the at least one adult stem cell marker in a control sample containing a known amount of the at least one adult stem cell type.

15. The method of claim 14, wherein the at least one adult stem cell type is selected from mesenchymal stem cells and hematopoietic stem cells.

16. The method of claim 14, wherein the at least one adult stem cell type is mesenchymal stem cells and the at least one adult stem cell marker is keratin 14 (K14).

17. The method of claim 14, wherein the at least one adult stem cell type is hematopoietic stem cells and the at least one adult stem cell marker is selected from tyrosine kinase with immunoglobulin-like and EGF-like domains 1 (TIE1) and CD48.

18. The method of claim 14, wherein the test sample is obtained from tissue selected from adipose tissue, amniotic fluid, aorta-gonad-mesonephros, bone, bone marrow, chorionic villi of the placenta, exfoliated deciduous teeth, fetal tissue, fetal liver, fetal lung, fetal spleen, hair follicles, liver, lung, myocardium, periosteum, peripheral blood, placenta, placental blood, skeletal muscle, skin, spleen, synovia, synovial fluid, tendon, umbilical cord blood, umbilical cord tissue.

19. The method of claim 14, wherein the method further comprises isolating mRNA from the test sample.

20. The method of claim 14, wherein the method further comprises using quantitative real time PCR to quantify the expression of the at least one adult stem cell marker.

21. The method of claim 14, wherein the expression of the at least one adult stem cell marker is quantified by hybridization of a nucleic acid obtained from the test sample to an array of nucleic acid probes.

22. The method of claim 14, wherein the amount of the at least one adult stem cell type in the test sample is determined by comparing the expression of the at least one adult stem cell marker in the test sample to a standard curve that relates the quantity of expression of the at least one adult stem cell marker to the proportion of the at least one adult stem cell type in a sample.

23. The method of claim 22, wherein the standard curve is created by a method comprising the steps of:
quantifying the expression of the at least one adult stem cell marker in at least one first control sample containing a known amount of the at least one adult stem cell type;
quantifying the expression of the at least one adult stem cell marker in at least one second control sample that does not contain a detectable amount of the at least one adult stem cell type; and
correlating the expression of the at least one adult stem cell marker to the amount of the at least one adult stem cell type.

24. The method of claim 23, wherein the at least one first control sample is obtained from cells selected from kidney cells and colon cells.

25. The method of claim 23, wherein the at least one second control sample is obtained from cells selected from adipocytes, brain astrocytes, cardiomyocytes, cenocytes, chondrocytes, cells of the visceral mesoderm (endothelial cells), endoderm cells, fibroblasts, glial cells, hepatocytes, keratinocytes, myoblasts, neural cells, neurons, osteoblasts, pancreatic β cells, skeletal myocytes, tenocytes, erythrocytes, platelets, basophils, eosinophils, neutrophils, granulocytes, dendritic cells, monocytes, macrophages, B cells, T cells, and NK cells.

26. The method of claim 23, wherein the expression of the at least one adult stem cell marker is quantified in at least ten first and at least ten second control samples.

27. The method of claim 14, wherein the patient has acute myelogenous leukemia, acute lymphoblastic leukemia, chronic lymphocytic leukemia, chronic myelogenous leukemia, multiple myeloma, non-Hodgkin lymphomas, diffuse large B cell lymphoma, mantel cell lymphoma, Hodgkin lymphoma, aplastic anemia, testicular germ-cell cancer, sickle cell
